(19) United States
(12) Patent Application Publication
(10) Pub. No.: US 2009/0169438 A1
(43) Pub. Date: Jul. 2, 2009

(54) AIR CLEANING APPARATUS

(75) Inventor: Wilhelmus Hendrikus Maria Bruggink, Drachten (NL)

Correspondence Address: PHILIPS INTELLECTUAL PROPERTY & STANDARDS, PO BOX 3001, BRIARCLIFF MANOR, NY, 10510-8001, US

(73) Assignee: KONINKLIJKE PHILIPS ELECTRONICS N.V., EINDHOVEN (NL)

(21) Appl. No.: 12/091,888

(22) PCT Filed: Oct. 24, 2006
(86) PCT No.: PCT/IB2006/053903

§ 371 Date: Aug. 6, 2008

(30) Foreign Application Priority Data
Oct. 26, 2005 (EP) 05109981.0

(51) Int. Cl. A61L 9/015; B01D 53/32; A61L 9/22; B03C 3/017

(52) U.S. Cl. 422/121; 422/123; 422/122

(57) ABSTRACT

The invention relates to an air cleaning apparatus, comprising a gas filtration section and a particle filtration section. The gas filtration section comprises a gas absorbing or adsorbing unit for trapping gaseous contaminants and a generator for generating reactive oxidizing species (ROS), suitable for oxidizing said gaseous contaminants. The particle filtration section comprises a precipitation unit, arranged to attract charged particles from passing air, and the (ROS) generator is arranged to charge said particles prior to their precipitation. Thus, the (ROS) generator fulfils a double function.

The invention relates to an air cleaning apparatus, more particularly an air cleaning apparatus for removing gasses (and accompanying odors) from indoor air.

Such air cleaning apparatuses are known. These known apparatuses make use of an absorbent or adsorbent material, such as activated carbon (AC), zeolite or some other porous material capable of trapping large amounts of gas. The apparatus may furthermore include a particle filter, such as a paper filter, a HEPA (High Efficiency Particle Arresting) filter or an electrete filter (featuring electrostatically charged fibres), for removing dust and other particles from the air, to prevent these particles from clogging or otherwise interfering with the absorbent or adsorbent material.

A problem with these known apparatuses is that, during use, the absorbent material becomes saturated with trapped gasses and therefore must be cleaned or replaced regularly. This is inconvenient and time-consuming. In WO 03/093734, it has been proposed to solve this problem by providing the air cleaning apparatus with an ionizing unit or an ozone-generating unit. Such units create an oxidative atmosphere, which causes the gasses trapped in the pores of the absorbent material to be oxidized into water molecules (H₂O) and carbon dioxide molecules (CO₂), thereby freeing up said pores.

A disadvantage of this known solution is that the unit for creating the oxidant atmosphere, which hereinafter will be called a ROS (Reactive Oxidizing Species) generator, adds to the total cost of the apparatus.

It is therefore an object of the invention to put to use said ROS generator more effectively, so as to further improve the cleaning performance of the air cleaning apparatus, thereby making the extra costs for the ROS generator worthwhile. To that end, an apparatus according to the invention is characterized by the features of claim 1.

In an air cleaning apparatus according to the invention, the ROS generator fulfils a double task. On the one hand, it produces an oxidative atmosphere, like in the prior art, which can regenerate the absorbent material, i.e. free its pores of trapped gasses. On the other hand, it imparts an electrostatic charge to particles, which are suspended in the air to be cleaned. Consequently, these particles can be readily removed from the air by means of a precipitation unit. Such a precipitation unit may comprise a number of elements, charged oppositely to the particles, which act therefore as ‘magnets’ that attract the particles.

Hence, the ROS generator cooperates with the precipitation unit to form an electro-static precipitation (ESP) filter. Such a filter may replace the aforementioned (mechanical) particle filters, offering several advantages. For instance, the pressure drop over the ESP filter is much lower than with mechanical filters, thanks to the relatively open structure of the ESP filter. Consequently, less power will be needed to force air past the ESP filter, which enables energy savings and may furthermore allow quieter operation.

It is noted that ESP filters in themselves are known. A known drawback of such filters is that they produce ozone while charging the particles to be filtered. Ozone may be a health hazard, which is the reason why usually attempts are made to minimize such ozone production. The current applicant, however, has had the inventive insight to turn the abovementioned drawback into an advantage, by combining the ESP filter with a gas-absorbent unit, which uses ozone to ‘clean’ its pores. Thus, there is no need to minimize the ozone production. On the contrary.

According to one aspect of the invention, the ROS generator may, for instance, comprise an ion generator, an ozone generator, a generator of radicals, in particular hydroxyl (OH), or a generator of any other reactive oxidizing gas. Such generators may be standard, commercially available components and may, for instance, rely on corona discharge technology. Of course, the ROS generator may involve other technology, for instance, based on chemicals and/or radiation, to create an oxidative atmosphere

When the ROS generator relies on corona discharge technology, the means for generating such corona discharge preferably comprise a series of corona wires, according to the features of claim 4. Such wires can generate a very homogeneous distribution of ROS over the gas-absorbing unit, which may contribute to a controlled, homogeneous regeneration of the absorbing material.

For similar reasons, the ROS generator is preferably disposed opposite the gas-absorbing unit, at some distance therefrom, according to the features of claim 5. Such a distance can help expose the gas-absorbing unit to an even more homogenously distributed ROS atmosphere, resulting in the aforementioned advantages.

Furthermore, the dimensions of the ROS generator are preferably selected to match those of the gas-absorbing unit, so that the generated ROS atmosphere covers the entire gas-absorbing unit, according to the features of claim 6. This will ensure that each portion of the gas-absorbing unit can regenerate properly.

According to an advantageous embodiment of the invention, the gas-absorbing unit may comprise one or more non-ox disable porous materials, according to the features of claim 7. Each material will feature a particular absorption affinity for a particular gas (which can be demonstrated by equilibrium absorption isotherms). Thus, for every gas to be removed from the air, the most suitable absorbent material or combination of materials can be selected.

According to another advantageous aspect of the invention, the absorbent material may be shaped according to the features of claim 8. Thanks to such a granular shape, the kinetics of the absorption process and/or the accessibility of the material can be enhanced, resulting in improved absorption performance.

Further advantageous embodiments of an air cleaning apparatus according to the invention are set forth in the dependent claims.

To explain the invention in further detail, an exemplary embodiment will be described of an air cleaning apparatus according to the invention, with reference to the accompanying drawings, wherein:

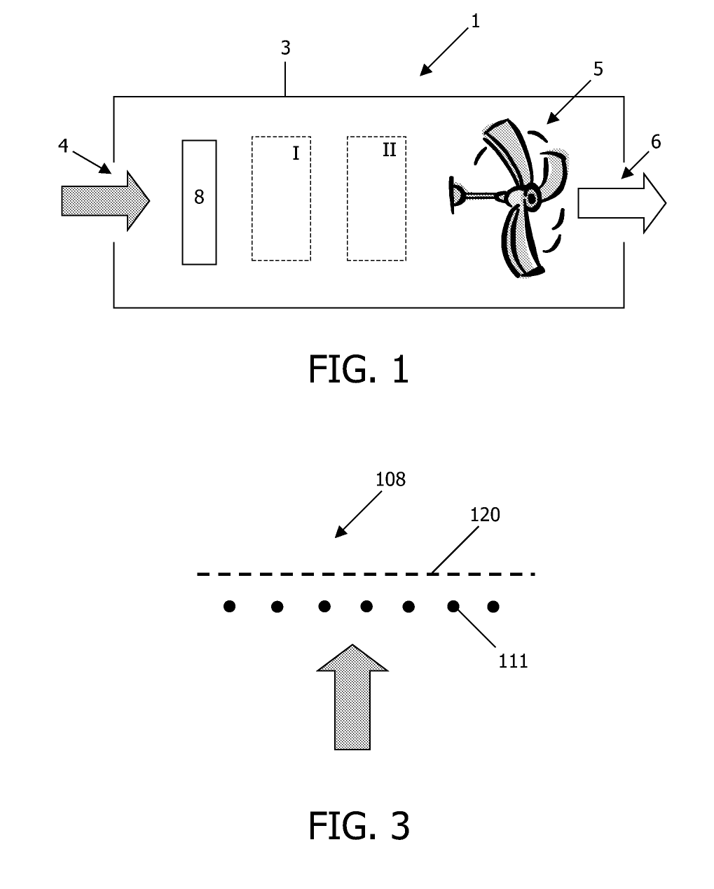

FIG. 1 schematically shows an air cleaning apparatus according to the invention;

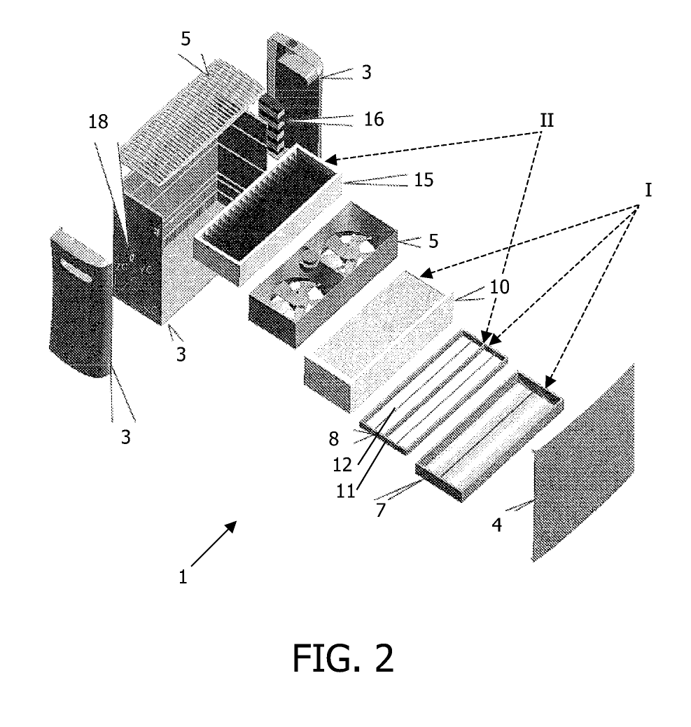

FIG. 2 shows an embodiment of the air cleaning apparatus according to FIG. 1, in exploded view; and

FIG. 3 shows one possible embodiment of a ROS generator for use in an air cleaning apparatus according to the invention.

In this description, the term ROS (Reactive Oxidizing Species) is understood to include, inter alia, charged ions, ion clusters, radicals, in particular hydroxyl radicals (OH-radicals), ozone or any other reactive oxidizing gas(es). ROS is usually generated electrically, but may be generated differently, for instance chemically or through radiation. Therefore, in this description, the term ROS generator is understood to mean each device, method and/or compound, capable of generating ROS, i.e. an oxidative atmosphere for gases. Furthermore, whenever in this description the term ‘absorbent’ is used, this may be replaced by ‘adsorbent’ and vice versa.

FIG. 1 schematically shows an air cleaning apparatus 1 according to the invention, comprising a particle filtration section I for filtering particles, such as for instance dust from passing air, and a gas filtration section II for filtering gasses (and accompanying odours) from passing air. The apparatus 1 furthermore comprises suction means 5, for instance a fan 5, for forcing air to be cleaned past said respective sections I, II, and a ROS generator 8, arranged to produce ROS (Reactive Oxidizing Species) and to charge particles in the passing air. It is noted that the specific arrangement as shown in FIG. 1 may vary. For instance, the sections I, II may (at least partly) overlap. The suction means 5 and/or ROS generator 8 may be positioned in between the sections I, II or upstream or downstream thereof. Alternatively, the ROS generator 8 may be configured to partly surround said sections I, II. The ensemble of components 5, 8 and sections I, II can be enclosed in a housing 3, having an inlet area 4 and an outlet area 6 for allowing air to be cleaned to enter and exit the apparatus 1.

FIG. 2 shows one possible embodiment of the air cleaning apparatus 1 according to FIG. 1. Corresponding parts have been denoted with corresponding reference numerals.

In this embodiment, the ROS generator 8 comprises a frame 11 equipped with two corona wires 12, configured to charge particles in passing air and to create an oxidative atmosphere. Besides this ROS generator 8, the particle filtration section I furthermore comprises a precipitation unit 10, provided with a number of collector elements, e.g. electrodes and/or plates (not visible in FIG. 2), that are imparted with a charge opposite to that of the charged particles. Consequently, when passing these collector elements, the particles will be attracted by the collector elements, and thus be removed from the air.

The particle filtration section I may furthermore comprise a mechanical pre-filter 7, which is preferably disposed near the inlet area 4, or at least upstream of the precipitation unit 10. The pre-filter 7 is preferably configured to filter relatively large particles from the air. Thus, said relatively large particles are prevented from clogging the precipitation unit 10, which may lengthen the lifetime of said precipitation unit 10 considerably or at least lengthen the time before the unit 10 needs to be cleaned. The pre-filter 7 can, for instance, be a (disposable) paper filter, an electrete filter (provided with electrostatically charged fibres) or any other suitable particle filter. Of course, in an alternative embodiment, more than one pre-filter may be used. Alternatively, the pre-filter 7 can be omitted.

The gas filtration section II comprises a gas-absorbing unit 15, which in the illustrated embodiment is configured as a pleated filter, filled with zeolite pellets. Of course, alternative embodiments are possible, wherein the filter may, for instance, be configured as having a honeycomb-structure. Also, alternative absorbing material can be applied, such as active alumina, micro-porous TiO2 or mixtures thereof.

As is best seen from FIG. 2, in assembled condition, the absorbing unit 15 and ROS generator 8 will be substantially aligned. Their dimensioning is such that the oxidative atmosphere generated by the ROS generator covers the entire gas absorbing unit 15. It can furthermore be seen that the gas absorbing unit 15 and the ROS generator 8 will be spaced at some distance from each other. All these features help to expose the gas-absorbing unit 15 to a substantially homogenous ROS distribution, which results in homogenous regeneration of the absorbing material. The space between the absorbing unit 15 and the ROS generator 8 may be used to install the fan 5 and precipitation unit 10, as illustrated in FIG. 2.

The air cleaning apparatus 1 further comprises voltage supply means 16 for supplying the ROS generator 8 and precipitation unit 10 with a suitable voltage. Furthermore, control electronics 18 may be provided for controlling specific operation parameters, such as for instance the fan speed and/or the voltage level supplied to the ROS generator 8 and the precipitation unit 10. Also, means may be provided for measuring the amount of particles collected in the precipitation unit 10. This can, for instance, be done by monitoring the condenser capacity of the collector elements of the precipitation unit 10. This capacity will change as more particles are collected. The measured information can be used to alarm a user when the precipitation unit 10 needs cleaning or replacement. Of course, comparable provisions may be provided for the pre-filter 7 and/or absorbing unit 15 (if, for instance, over time the pores become clogged with small particles).

The above-described air cleaning apparatus 1 operates as follows. Once activated, fan 5 will suck surrounding air into the apparatus 1, via inlet area 4. The air will then successively pass the pre-filter 7, where it is freed of relatively large particles, the ROS generator 8, where the remaining particles are electrically charged, the precipitation unit 10, where it will leave behind the charged particles at the oppositely charged collector elements, and finally the gas absorbing unit 15, where it will be freed of undesired gasses, which will stay behind in pores of the absorbing material. There the gasses will oxidize into water molecules and carbon dioxide molecules under the influence of the ROS produced by the ROS generator 8.

By way of illustration only, the following example is given of a test carried out by the applicant. The given values should in no way be construed as limiting the scope of protection. In the embodiment according to FIG. 2, the two corona wires 11 were made of tungsten, each having a diameter of 0.08 mm. The corona voltage was set to 7.9 kV. This resulted in an amount of ROS ranging from approximately 200 to 400 micrograms ozone per hour at an air speed of 2 meters per second. Furthermore, the voltage at the precipitation unit 10 was set to 4.7 kV. This resulted in an initial particle trapping efficiency of almost 100% for particles with a dimension of 0.3 μm. The gas-absorbing unit 15 was provided with pleated granular zeolite, arranged in a bed having a length of 400 mm, a width of 150 mm and a thickness of 10 mm. When air mixed with toluene (a VOC: volatile organic compound) was passed through this unit 15, a one-pass removal efficiency was observed ranging from approximately 65% to 80%, which corresponds to an overall concentration reduction of 800 μg/m³ to 92 μg/m³.

FIG. 3 shows an alternative embodiment of a ROS generator 108, suitable for application in an air cleaning apparatus 1 according to the invention. In this embodiment, the ROS generator 108 comprises a series of corona wires 111, extending substantially parallel to each other at some distance from an earthed gauze 120. The arrow indicates the direction of the passing air to be cleaned. By varying the distance between the wires and said gauze and/or the distance between the respective wires, one can influence the critical corona voltage. Preferably, a high corona voltage is applied. This results in a high corona current, which in turn results in more gas molecules splitting up, leading to a more oxidizing atmosphere, which of course in the present invention is beneficial for the regeneration of the absorbing unit 15. It is furthermore preferred to use a negative corona. A negative corona charges the particles as effectively as a positive corona, yet produces a more oxidizing atmosphere. Also, it is preferred to use relatively thin corona wires, having a diameter which is preferably smaller than 100 microns, and which are preferably made of tungsten instead of, for instance, stainless steal. This too will help to produce a more oxidizing atmosphere. For the same reason it is preferred to use corona wires having a relatively rough surface. Finally, it is preferred to configure the corona section in such way that air passing this section is exposed to corona during a relatively long time. Thus, charging of particles and formation of ROS will be enhanced.

According to another embodiment, the ROS generator may comprise an ion wind generator. The ion wind created by such a generator can drive air through the air cleaning apparatus, thereby offering the advantage that the suction means 5 (fan) can be dispensed with. This results in an air cleaning apparatus that can operate extremely quietly.

In yet another embodiment, the gas filtration section and particle filtration section may be combined by covering the collector plates of the precipitation unit 10 with a layer of a non-oxidizing adsorbent, for instance a zeolite slurry.

The invention is not in any way limited to the exemplary embodiments presented in the description and drawing. All combinations (of parts) of the embodiments shown and described in this description are explicitly understood to be incorporated within this description and are explicitly understood to fall within the scope of the invention. Moreover, many variations are possible within the scope of the invention, as outlined by the appended claims.

1. Air cleaning apparatus comprising a gas filtration section and a particle filtration section, said gas filtration section comprising a gas absorbing or adsorbing unit for trapping gaseous contaminants and a generator for generating reactive oxidizing species (ROS) that are suitable for oxidizing said gaseous contaminants, characterized in that the particle filtration section comprises a precipitation unit, arranged to attract charged particles from passing air, and the ROS generator is arranged to charge said particles.

2. Air cleaning apparatus according to claim 1, wherein the ROS generator comprises at least one generator from the following list of generators: an ion generator, a generator of radicals, in particular hydroxyl radicals, a generator of ozone or any other reactive oxidizing gas.

3. Air cleaning apparatus according to claim 1, wherein the ROS generator comprises means for generating a corona discharge.

4. Air cleaning apparatus according to claim 3, wherein the means for generating a corona discharge comprise a series of corona wires.

5. Air cleaning apparatus according to claim 1, wherein the ROS generator is disposed opposite the gas-absorbing unit, at some distance therefrom.

6. Air cleaning apparatus according to claim 1, wherein the dimensions of the ROS generator are selected such that the generated ROS atmosphere covers substantially the entire air passage area of the gas-absorbing unit.

7. Air cleaning apparatus according to claim 1, wherein the gas absorbing unit includes a non-ox disable porous material, such as for instance natural or synthetic zeolite, active alumina, micro-porous TiO2.

8. Air cleaning apparatus according to claim 7, wherein the non-ox disable porous material is granular shaped and contained in a suitable structure, for instance a honeycomb structure.

9. Air cleaning apparatus according to claim 1, furthermore comprising an amount of oxidizing material, such as for instance activated carbon, disposed downstream of the gas absorbing unit, and arranged to eliminate or neutralize residual ROS.
